(19) United States
(12) Patent Application Publication
(10) Pub. No.: US 2020/0289048 A1
(43) Pub. Date: Sep. 17, 2020

(54) TOILET WITH SOFT TISSUE HARDNESS TESTER

(71) Applicants: David R. Hall, Provoq, UT (US); Joshua Larsen, Spanish Fork, UT (US); Jared Reynolds, Pleasant Grove, UT (US); K. Jeffrey Campbell, Spanish Fork, UT (US); Travis Niederhauser, Mapleton, UT (US); Daniel Hendricks, Provo, UT (US); Steven J.M. Butala, Provo, UT (US); Vivek Garg, Murray, UT (US)

(72) Inventors: David R. Hall, Provoq, UT (US); Joshua Larsen, Spanish Fork, UT (US); Jared Reynolds, Pleasant Grove, UT (US); K. Jeffrey Campbell, Spanish Fork, UT (US); Travis Niederhauser, Mapleton, UT (US); Daniel Hendricks, Provo, UT (US); Steven J.M. Butala, Provo, UT (US); Vivek Garg, Murray, UT (US)

(21) Appl. No.: 16/352,130

(22) Filed: Mar. 13, 2019

(51) Int. Cl. A61B 5/00; A47K 13/24; A47K 17/02

(52) U.S. Cl. CPC A61B 5/442; A47K 13/24; A47K 17/028; A61B 2562/168; A61B 5/6891; A61B 2562/0247; A61B 5/4519

(57) ABSTRACT

The medical toilet includes one or more sensors for measuring hardness of a soft tissue in contact with the sensor. The one or more sensors may be positioned on the toilet seat, toilet lid, or on a foot scale which extends from the base of the medical toilet. The sensors may include durometers, fluid-filled bubbles with pressure sensors on them, or an inflatable tube with pressure sensors on it. The measurements may be transmitted to a controller which may store and analyze the data. Changes in tissue hardness over time may be tracked and reported. The sensors may identify changes in the hardness of skin, adipose tissue, and muscle. These measurements may be useful to identify changes in skin thickness, body fat, muscle tone, tumors and other masses.

### BACKGROUND

### Field of the Invention

This disclosure relates to durometers and devices which collect measurements hardness of a substance, specifically a tissue, for use in monitoring health and wellness.

### Background of the Invention

Durometer is a hardness test of a material. It measures the depth of an indentation in the material created by a defined force on a standardized presser foot. The depth of the indentation is inversely related to the hardness of the material. The depth of the indentation is dependent on the following variables: hardness of the material, viscoelastic properties of the material, the shape of the presser foot, and the duration of the test.

Durometer is typically used to measure the hardness of synthetic materials, for example, textiles and synthetic polymers. Some of the materials commonly measured with durometer are quite soft, for example, blown foam products, sponge rubber, medical pads, and gel-like materials. However, it is useful to know the hardness of soft tissue as well. Changes in the hardness of a soft tissue are also relevant from a health and well-ness perspective. For example, measuring how firm a person's skin or muscle is may be an indicator of the person's health and fitness. If the person increases muscle mass in an area of the body, the durometer would increase in that area. Alternatively, if the person increases body fat in an area, the durometer would decrease in that area. Skin calluses would result in an increased durometer in the area of the callus. Solid tumors may cause an increase in durometer.

Toilets are used daily to deposit bodily waste. Recently, medical toilets have emerged which collect measurements which are relevant to a user's health status. By collecting these measurements while a user is seated on the toilet, the measurements are collected on a regular basis and the user is not significantly inconvenienced. A medical toilet which includes one or more tissue hardness sensors positioned at strategic locations may provide a repeatable non-intrusive way to measure body durometer at several points.

### BRIEF SUMMARY OF THE INVENTION

We disclose a medical toilet which includes one or more sensors on at least one surface of the toilet. Each sensor measures the hardness of a soft tissue of a medical toilet user with which it comes in contact. The measurement of tissue hardness may be used to determine the hardness of skin, adipose tissue, and muscle. Masses, for example, tumors, could be identified and their growth tracked by repeatedly measuring their hardness using the disclosed medical toilet.

The sensors may be located on one or more of the toilet seat, toilet lid, and a foot scale which extends from a base of the medical toilet. A user may sit on the medical toilet just as the user may sit on a traditional western style toilet. The sensors on the toilet seat may come in contact with the user's thighs and/or buttocks. The user may lean back against the toilet seat causing the sensors to contact the user's back. The user may put both feet on the foot scale causing the sensors to come in contact with each of the user's feet.

The sensors may include a durometer which may measure hardness of soft tissue according to standard durometer scales which may include Shore 00, Shore A, and Shore D scales. Each durometer may include an indenter and a spring. The indenter may be in the shape of a rod or a pointed cone. Each durometer may be in electronic communication with a controller. The controller may include non-transitory computer readable media which may store instructions for storing and analyzing measurements collected by each durometer to determine the hardness of a soft tissue with which the durometer has come in contact.

The sensors may also be one or more fluid-filled bubbles located on a surface of the medical toilet. The fluid in the fluid-filled bubbles may be either liquid or gaseous. Each fluid-filled bubble may have a pressure sensor disposed on it. The pressure sensors may be in electronic communication with a controller. The controller may include non-transitory computer readable media which may store instructions for storing and analyzing measurements collected by each pressure sensor to determine the hardness of a soft tissue with which the fluid-filled bubble has come in contact.

The toilet seat may include an inflatable tube which may be inflated by a fluid, either liquid or gaseous. The medical toilet may include a pump which may drive the fluid into the inflatable tube. A plurality of pressure sensors may be disposed along the length of the inflatable tube. A user may sit on the toilet seat and the inflatable tube causing the inflatable tube and pressure sensors thereon to come in contact with the user's thighs and/or buttocks. The pressure sensors may be in electronic communication with a controller. The controller may include non-transitory computer readable media which may store instructions for storing and analyzing measurements collected by each pressure sensor to determine the hardness of a soft tissue with which the inflatable tube has come in contact.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings.

### Finish Description of Drawings

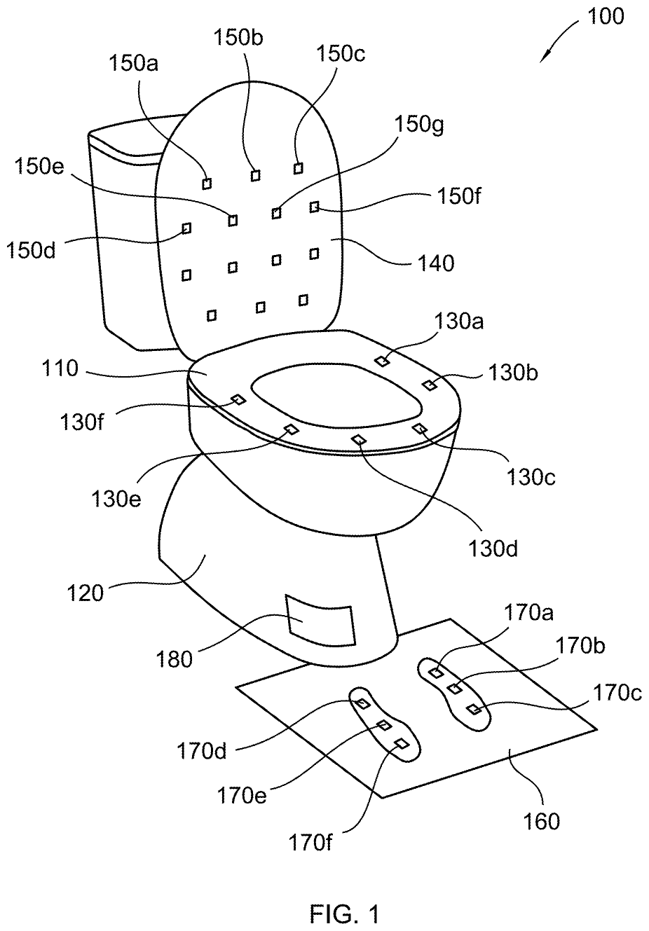

FIG. 1 is a perspective view of a medical toilet including durometers on various surfaces according to an embodiment of the disclosure.

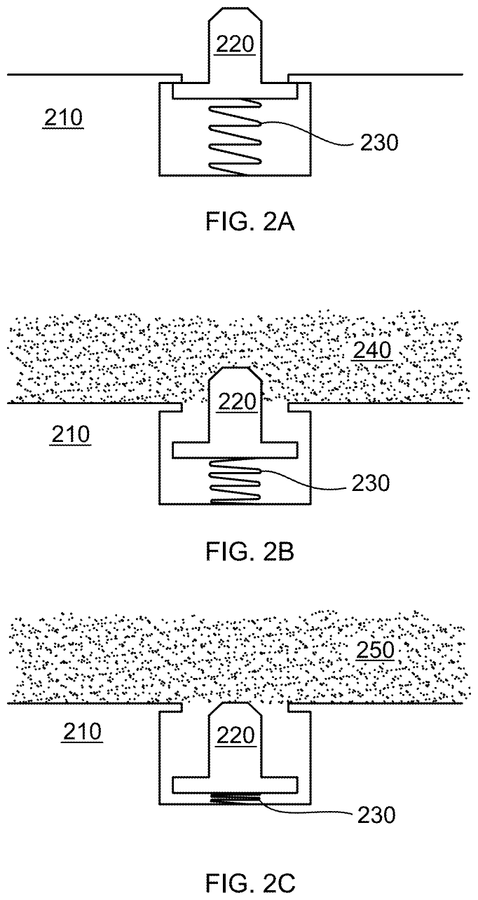

FIGS. 2A, 2B, and 2C are schematic views of a first embodiment of a durometer within a surface of the medical toilet in contact with no tissue, a softer tissue, and a harder tissue respectively.

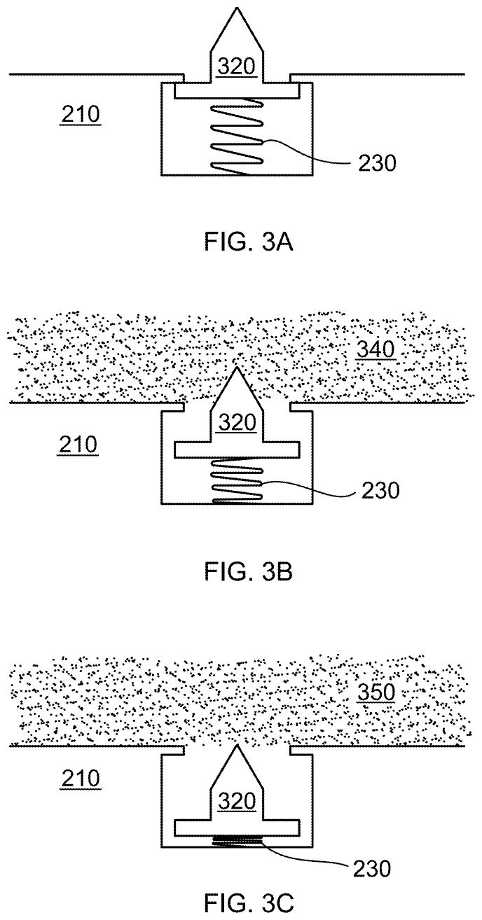

FIGS. 3A, 3B, and 3C are schematic views of a second embodiment of a durometer within a surface of the medical toilet in contact with no tissue, a softer tissue, and a harder tissue respectively.

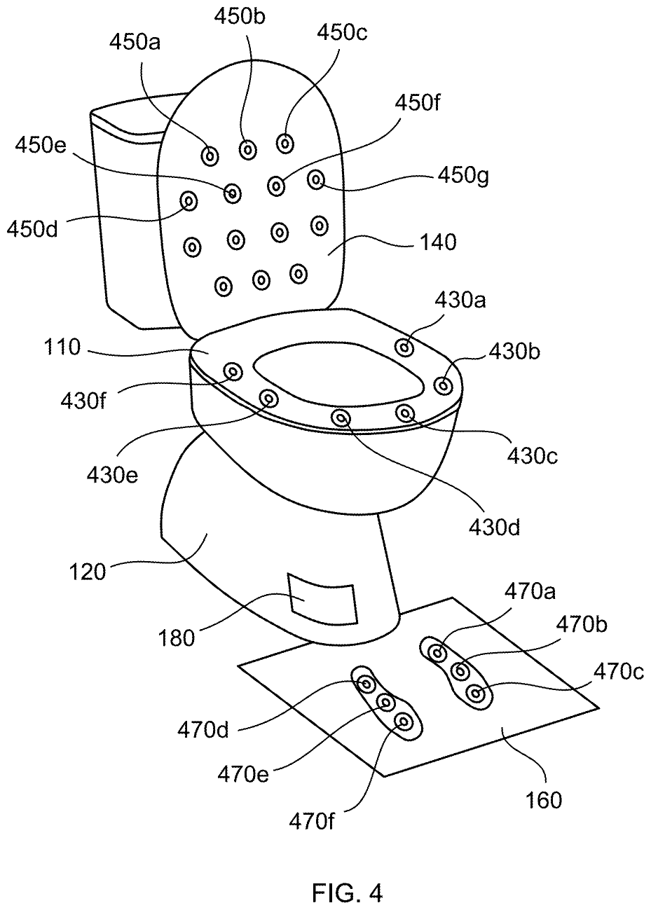

FIG. 4 is a perspective view of a medical toilet which includes fluid-filled bubbles on various surfaces according to an embodiment of the disclosure.

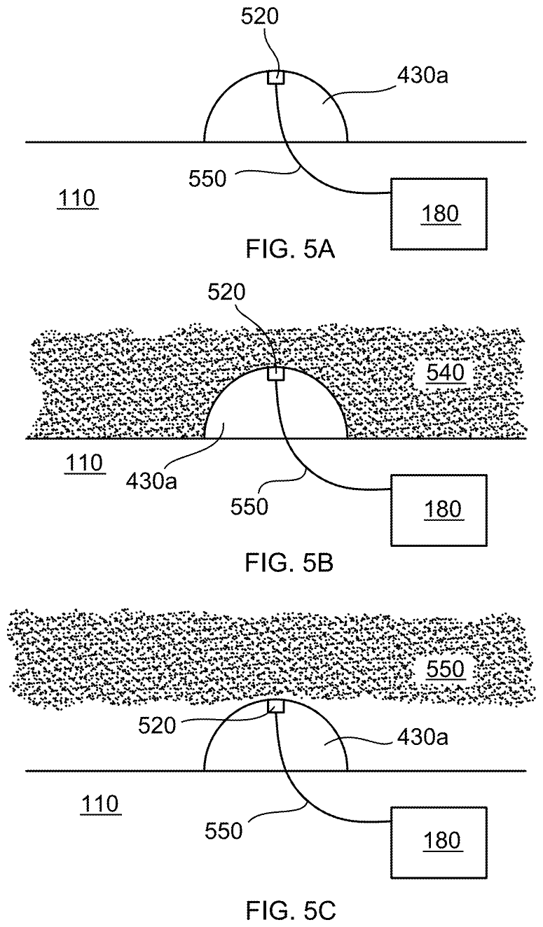

FIGS. 5A, 5B, and 5C are schematic views of a fluid-filled bubble on a surface of the medical toilet in contact with no tissue, a softer tissue, and a harder tissue respectively.

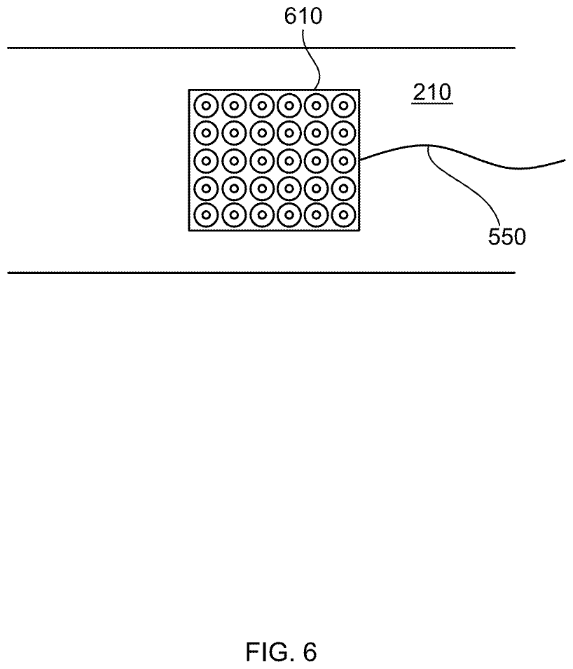

FIG. 6 is a schematic view of an array of fluid-filled bubbles according to an embodiment of the disclosure.

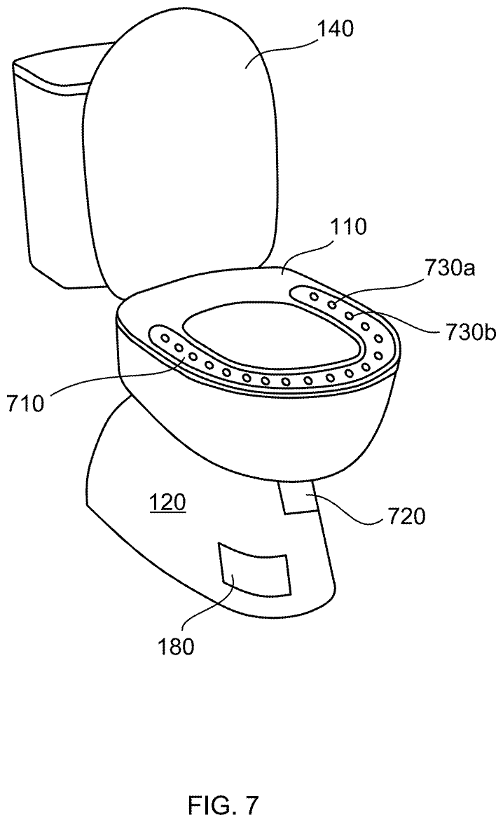

FIG. 7 is a perspective view of a medical toilet which includes an inflatable tube on the toilet seat according to an embodiment of the disclosure.

### DETAILED DESCRIPTION OF THE INVENTION DEFINITIONS

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “user” means the individual who comes in contact with the medical toilet disclosed herein and/or who deposits bodily waste into the medical toilet disclosed herein.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

The disclosed medical toilet creates a repeatable, non-intrusive, and passive way to measure the hardness of soft tissue at one or several points on a user's body. By placing the sensors in one or in several spots on the toilet seat, on the toilet lid, or on a foot pad, the medical toilet may measure the firmness of the skin, muscle, or other tissues on a user's thighs, buttocks, back, and/or feet. Typically, a user sits on a toilet seat in the same position during each use. Consequently, if repeated measurements are collected over time, it is possible to see changes in the hardness measurement of a particular tissue.

We disclose a medical toilet which is a platform for one or many sensors which measure hardness of a tissue. The medical toilet may include a user identification system which may include a password, a key card, or a biometric measurement device. When the user is identified, soft tissue hardness data may be gathered and changes in hardness may be monitored over time. When a user is building more muscle mass or increasing muscle tone, the disclosed device may measure a higher tissue hardness in certain areas. If a user has excessive calluses on his or her feet, the device may measure an increased skin hardness as the calluses develop. If a user is increasing in body fat, the device may measure a lower tissue hardness in fatty areas of the user's body.

The disclosed medical toilet may include one or a plurality of durometer sensors which may be placed on or within one or more surfaces of the medical toilet. When the user is identified, data may be gathered and changes over time may be detected. For example, when someone is building more muscle mass or tone, a higher durometer measurement in certain areas may be measured. If someone has excessive calluses on their feet, an increased durometer measurement on the user's feet may be collected as the calluses grow. If the user is experiencing an increase in body fat, the durometer measurement in fatty areas may drop.

A toilet is an ideal location to position the hardness sensors according to the disclosure because a user removes clothing while using the toilet and the hardness sensor may be placed in contact with areas of skin that are normally not exposed. People also tend to sit on the toilet in a similar fashion each time they use it. Consequently, repeated hardness measurements of the same area and/or same tissue may be collected so that change over time may be monitored.

The disclosed medical toilet may also include additional sensors, for example, in the seat, to assess the position in which a user sitting. If the posture and position of the user is known, algorithms which process collected data according to the known posture and position may be stored on a controller which is in electronic connection the hardness sensors. This may result in improved accuracy and repeatability of the hardness data.

In some embodiments, the hardness sensors may be disposed on a toilet seat or toilet lid. In other embodiments, the hardness sensors may be disposed on a bathroom scale or a footpad which detects a pressure pattern.

In some embodiments, the bathroom scale or footpad may be positioned in front of the toilet. In these embodiments, the hardness sensors may be used to collect measurements of the hardness of a user's feet. For example, callouses, bunions, or tumors on a user's feet may be detected. In some embodiments, the hardness sensors on the bathroom scale or footpad may be used with other hardness sensors on the toilet and/or with other devices associated with a medical toilet. These other devices associated with a medical toilet may collect additional measurements that may be relevant to a user's health and well-being. Alternatively, the hardness sensors on the bathroom scale or footpad may be used independently from a toilet.

In some embodiments, hardness sensors may be positioned on the underside of a toilet lid and so that they press against a user's back when the user leans back while seated on the toilet. By placing a plurality of hardness sensors at various points on the toilet lid, hardness measurements may be collected from a plurality of points on a user's back. Alternatively, the sensors may be positioned on a platform which moves the hardness sensors toward the user's back as them. An example of such a platform is disclosed in U.S. patent application Ser. No. 16/272,206 filed on Feb. 11, 2019 which is hereby incorporated by reference in its entirety.

In an example, the hardness sensors include one or more durometers. The one or more durometers may each include an indenter and a spring. In some such embodiments, the indenter may be a rod and, in others, a pointed cone. The one or more durometers may be configured to measure the hardness of a soft tissue, for example, skin, muscle, and adipose tissue. The one or more durometers may measure soft tissue hardness using one or more of the following durometer scales: Shore OO, Shore A, and Shore D.

In some embodiments, the one or more durometers may be disposed on the toilet seat on the medical toilet. While some embodiments may include a single durometer which contacts a user's thigh or buttocks when the user is seated on the toilet seat, others may include multiple durometers which contact the user's thigh(s) and/or buttocks at multiple locations. Hardness of the skin on the user's thigh, body fat on the thigh, and muscle tone, for example, in the user's quadriceps, may be determined using the durometer measurement. How easily a user's skin may tear as well as gain or loss of muscle tone may be monitored using this medical toilet. A pathological mass may also be detected in the user's thigh because the mass may have a different hardness than surrounding tissue.

The one or more durometers may be disposed on a toilet lid. A user may sit on the toilet seat of the medical toilet and lean back against the toilet lid. The one or more durometers may detect hardness of the skin on the user's back which may be an indicator of muscle hardness, fat deposits, and masses on the user's back as described above with regard to the thigh.

The medical toilet may include a foot scale which may be extend outward from the base of the toilet on the floor. A user seated on the medical toilet may place his or her feet on the foot scale. The one or more durometers may detect the hardness of the user's skin on the bottom of the user's feet. In doing so, the medical toilet may detect calluses, bunions, or other masses on the user's feet. In embodiments which include a plurality of durometers, the indenters within the durometers may be actuated to provide pressure at points to perform acupuncture or massage the feet.

The medical toilet may include a controller which may be in electronic connection with the at least one durometer. The controller may include non-transitory computer readable media which stores instructions for storing the measurements of tissue hardness that the one or more durometers collect. The non-transitory computer readable media may include instructions for analyzing the measurements of tissue hardness collected by the one or more durometers to determine the hardness of a soft tissue from which the measurements were taken.

In addition to the embodiments of the medical toilet described above which include durometers as the hardness sensors, other embodiments may include at least on fluid-filled bubble which is configured to measure a hardness of a soft tissue. The at least on fluid-filled bubble may be disposed on one or more surfaces of the medical toilet and protrude from the one or more surfaces. Each fluid-filled bubble may include a pressure sensor which detects the hardness of a soft tissue.

The fluid within each fluid-filled bubble may be gas or liquid. In an example, the fluid is water. In another example, the fluid is air.

The one or more fluid-filled bubbles may be disposed on the toilet seat on the medical toilet. While some embodiments may include a single fluid-filled bubble which contacts a user's thigh or buttock when the user is seated on the toilet seat, others may include multiple fluid-filled bubbles which contact the user's thighs and/or buttocks at multiple locations. Hardness of the skin, body fat, and muscle tone, for example, in the user's quadriceps or gluteus maximus, may be determined using the fluid-filled bubble measurement. How easily a user's skin may tear as well as gain or loss of muscle tone may be monitored using this medical toilet. Masses may also be detected in the user's thigh or buttock because the mass may have a different hardness than surrounding tissue.

The one or more fluid-filled bubbles may be disposed on a toilet lid. A user may sit on the toilet seat of the medical toilet and lean back against the toilet lid. The one or more fluid-filled bubbles may detect hardness of the skin on the user's back. Fat deposits, muscle hardness, and masses on the user's back as described above with regard to the thigh and buttock.

The medical toilet may include a foot scale which may be extend outward from the base of the toilet on the floor. A user seated on the medical toilet may place his or her feet on the foot scale which may include one or more fluid-filled bubbles as disclosed herein. The one or more fluid-filled bubbles may detect the hardness of the user's skin on the bottom of the user's feet. In doing so, the medical toilet may detect calluses. The fluid-filled bubbles may also identify bunions or other masses on the user's feet.

The medical toilet may include a controller which may be in electronic connection with the at least one fluid-filled bubble or, more specifically, the pressure sensor on each of the at least one fluid-filled bubbles. The controller may include non-transitory computer readable media which stores instructions for storing the measurements of tissue hardness that the one or more fluid-filled bubbles, and more specifically, the pressure sensors thereon, collect. The non-transitory computer readable media may include instructions for analyzing the measurements of tissue hardness collected by the one or more fluid-filled bubbles to determine the hardness of a soft tissue from which the measurements were taken and for calculating trends and changes in the tissue hardness.

In some embodiments, the medical toilet includes a plurality of fluid-filled bubbles. The fluid-filled bubbles may be positioned in an array. The array may be in electronic connection with a controller either by individual connections with each fluid-filled bubble or through a single connection from the array as a whole.

Similar to the fluid-filled bubbles, some embodiments of the medical toilet include an inflatable tube in connection with the toilet seat. The inflatable tube may be disposed on the surface of the toilet seat or embedded within the toilet seat. A pump may be in fluid communication with the inflatable tube and may inflate the inflatable tube with a liquid or gaseous material. In some embodiments, the liquid material may be water. In some embodiments, the gaseous material may be air.

A plurality of pressure sensors may be disposed on or within the inflatable tube. Each of the plurality of pressure sensors may be in electronic communication with a controller. The controller may be within the medical toilet or associated through wireless connections.

A user may be seated on the toilet seat, thereby coming in contact with the inflatable tube. Sections of the inflatable tube may indent into the user's legs or buttocks similar to how the fluid-filled bubbles press into a user's tissue according to the hardness of the tissue. The harder the tissue, the more pressure the pressure sensors in contact with the tissue will sense. Each of the plurality of pressure sensors may send their pressure readings to the controller which may include non-transitory computer readable media. The non-transitory computer readable media may include instructions for analyzing the measurements of tissue hardness collected by the plurality of pressure sensors on the inflatable tube to determine the hardness of a soft tissue from which the measurements were taken.

Referring now to the drawings, FIG. 1 illustrates medical toilet 100, a medical toilet according to an embodiment of the disclosure. Medical toilet 100 includes toilet seat 110 which is mounted on toilet base 120. Durometers 130a-f are shown on toilet seat 110. A user may sit on toilet seat 110 and the user's thighs and buttocks may come in contact with durometers 130a-f. Durometers 130a-f may collect measurements of the hardness of the user's skin, adipose tissue, and muscle as well as any masses which may be present on the user's thighs and buttocks.

Medical toilet 100 further includes toilet lid 140 which includes durometers 150a-g. Additional durometers are shown on toilet lid 140 but are not labeled for purposes of clarity. A user may sit on medical toilet 100 and lean back against toilet lid 140. The user's back may come in contact with durometers 150a-g which may collect measurements of the hardness of the user's skin, adipose tissue, and muscle on the user's back as well as any masses which may be present on the user's back.

Medical toilet 100 includes foot scale 160 which includes durometers 170a-f. A user may place a left foot on durometers 170a-c and a right foot on durometers 170d-f. Durometers 170a-f may collect measurements of the hardness of the user's skin on the bottoms of the feet to detect calluses, as well as masses such as tumors, cysts, and bunions.

Durometers 130a-f, 150a-g, and 170a-f may be in electronic communication with controller 180. Non-transitory computer readable medium on controller 180 may store the data the durometers collect. The non-transitory computer readable medium may also store instructions for analyzing the data to provide an indicator of hardness and identify changes in hardness that have occurred over the time that multiple measurements have been collected.

FIGS. 2A-C illustrate a durometer which may be included in the medical toilet disclosed herein. The durometer is positioned on surface 210 which may be a toilet seat, toilet lid, foot scale, or other surface on a medical toilet according to the disclosure. The durometer includes indenter 220 which is a rod in mechanical communication with spring 230. FIG. 2A illustrates indenter 220 in a fully extended position with no tissue or other material positioned against it to compress spring 230. FIG. 2B illustrates the durometer of FIG. 2A with soft tissue 240 pressed against indenter 220. Soft tissue 240 may be a user's thigh, back, foot, or other body part. Indenter 220 is partially retracted and spring 230 is partially compressed. Indenter 220 extends into soft tissue 240 to an extent that is a function of the hardness of soft tissue 240. FIG. 2C illustrates the durometer of FIG. 2A with soft tissue 250 pressed against indenter 220. Soft tissue 250 has a greater hardness measurement than soft tissue 240. This hardness is indicated by indenter 220 being shown fully retracted and spring 230 being fully compressed.

FIG. 3A-C is a durometer which may be included in the medical toilet disclosed herein. The durometer of FIGS. 3A-C is similar to that of FIGS. 2A-C except that indenter 320 is a pointed cone instead of a rod in mechanical communication with spring 230. FIG. 3A shows indenter 320 in a fully extended position with no tissue or other material positioned against it. As disclosed above in the description of FIGS. 2A-C, surface 210 may be a toilet seat, toilet lid, foot scale, or other surface on a medical toilet according to the disclosure. FIG. 3B illustrates the durometer of FIG. 3A with soft tissue 340 pressed against indenter 320. Soft tissue 340 may be a user's thigh, back, foot, or other body part. Indenter 320 is partially retracted and spring 230 is partially compressed. Indenter 320 extends into soft tissue 340 to an extent that is a function of the hardness of soft tissue 240. FIG. 2C illustrates the durometer of FIG. 2A with soft tissue 350 pressed against indenter 220. Soft tissue 350 has a greater hardness measurement than soft tissue 340. This hardness is indicated by indenter 320 being shown fully retracted and spring 230 being fully compressed.

FIG. 4 illustrates medical toilet 400 which is similar to medical toilet 100 of FIG. 1. Both medical toilets 100 and 400 include toilet seat 100, toilet base 120 and toilet seat 140. Foot scale 160 extends from toilet base 120 and controller 180 is positioned within toilet base 120 in both medical toilets.

In contrast with medical toilet 100, toilet seat 110 of medical toilet 400 includes fluid-filled bubbles 430a-f. Toilet seat 110 of medical toilet 400 includes fluid-filled bubbles 430a-f. As explained in more detail in the discussion of FIGS. 5A-C, the fluid-filled bubbles measure the hardness of a soft tissue. The hardness of a soft tissue as measured by the fluid-filled bubbles may be used in the same way as the hardness measured by a durometer as described above. A user may sit on toilet seat 110 and the user's thighs and/or buttocks may come in contact with fluid-filled bubbles 430a-f. Fluid-filled bubbles 430a-f may collect measurements of the hardness of the user's skin, adipose tissue, and muscle as well as any masses which may be present on the user's thighs and/or buttocks.

Toilet lid 140 of medical toilet 400 includes fluid-filled bubbles 450a-g. Additional fluid-filled bubbles are present on toilet lid 140 but not labeled for purposes of clarity. A user may sit on medical toilet 400 and lean back against toilet lid 140. The user's back may come in contact with fluid-filled bubbles 450a-g which may collect measurements of the hardness of the user's skin, adipose tissue, and muscle on the user's back as well as any masses which may be present on the user's back.

Foot scale 160 includes fluid-filled bubbles 470a-f. A user seated on medical toilet 400. A user may place a left foot on fluid-filled bubbles 470a-c and a right foot on fluid-filled bubbles 470d-f. Fluid-filled bubbles 470a-f may collect measurements of the hardness of the user's skin on the bottoms of the feet, as well as masses such as calluses and bunions.

Fluid-filled bubbles 430a-f, 450a-g, and 470a-f may be in electronic communication with controller 180. Non-transitory computer readable medium on controller 180 may store the data the fluid-filled bubbles collect. The non-transitory computer readable medium may also store instructions for analyzing the data to provide an indicator of hardness and identify changes in hardness that have occurred over the time that multiple measurements have been collected.

FIGS. 5A-C illustrate fluid-filled bubble 430a which is one of the fluid-filled bubbles on medical toilet 400 first presented in FIG. 4. Fluid filled bubble 430a protrudes from the surface of toilet seat 110 of medical toilet 400. Pressure sensor 520 is disposed on fluid-filled bubble 430a. Electrical connector 550 provides an electrical connection between pressure sensor 520 and controller 180. While electrical connector 550 is shown as an electrical wire, in some embodiments, the electrical connection may be wireless.

FIG. 5B shows soft tissue 540 in contact with fluid-filled bubble 430a which extends into soft-tissue 540. Pressure sensor 520 measures the pressure that soft tissue 540 exerts on fluid-filled bubble 430a. This pressure measurement is an indicator of the hardness of soft tissue 540.

FIG. 5C shows fluid-filled bubble 430a as it measures the hardness of soft tissue 550. Because soft tissue 550 is a harder tissue than soft tissue 540, fluid-filled bubble 430a does not extend into soft tissue 550. Pressure sensor 520 measures the pressure that soft tissue 550 applies to fluid-filled bubble 430a and transmits the measurement electronically to controller 180 through electrical connector 550.

FIG. 6 illustrates another embodiment of a plurality of fluid-filled bubbles positioned in array 610 on surface 210 of the medical toilet. Other than being organized in an array, the fluid filled bubbles may be as shown in FIGS. 5A-C. Electrical connector 550 transmits measurements collected by array 610 to a controller for storage and analysis. When positioned as an array, the fluid-filled bubbles in array 610 collect hardness of a soft tissue over a larger area relative to single fluid-filled bubbles.

FIG. 7 illustrates yet another embodiment of the medical toilet which includes inflatable tube 710 on or within toilet seat 110. Pump 720 may fill inflatable tube 710 with a liquid or gaseous material. In an example, the liquid material is water. In another example, the gaseous material is air. A plurality of pressure sensors is shown along inflatable tube 710. Pressure sensors 730a and 730b are labeled while other pressure sensors are not labeled for purposes of clarity.

A user may be seated on toilet seat 110 with the user's thighs and/or buttocks in contact with inflatable tube 710. Depending on the hardness of the user's skin, fat, and muscle making up the user's thighs and/or buttocks, inflatable tube 710 may extend into the user's tissue according to the hardness of the tissue. The pressure sensors in contact with the tissue (which may include pressure sensors 730a-b) may collect a pressure reading and transmit the reading to the controller. Non-transitory computer readable media on the controller may store and analyze the pressure readings to determine the hardness of the tissue.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A medical toilet comprising:
a. a toilet seat; and
b. at least one durometer, the at least one durometer comprising an indenter, and a spring;
c. wherein each of the at least one durometer is disposed on or in a surface of the medical toilet;
d. wherein the at least one durometer is configured to measure a hardness of a soft tissue.

2. The medical toilet of claim 1, wherein the at least one durometer consists of a plurality of durometers.

3. The medical toilet of claim 2, wherein the surface of the medical toilet comprises a surface of the toilet seat; and wherein the plurality of durometers is disposed on the surface of the toilet seat.

4. The medical toilet of claim 1, wherein the surface of the medical toilet comprises a surface of the toilet seat.

5. The medical toilet of claim 1, further comprising a toilet lid, wherein the surface of the medical toilet comprises a surface of the toilet lid.

6. The medical toilet of claim 1, further comprising a foot scale, wherein the surface of the medical toilet comprises a surface of the foot scale.

7. The medical toilet of claim 1, further comprising a controller, wherein the controller is in electronic connection with the at least one durometer; and wherein the controller comprises non-transitory computer readable media, the non- transitory computer readable media comprising instructions for storing and analyzing measurements collected by the at least one durometer to determine the hardness of a soft tissue.

8. The medical toilet of claim 1, wherein the at least one durometer is configured to measure the hardness of the soft tissue using one or more of the following list of durometer scales: Shore OO, Shore A, and Shore D.

9. The medical toilet of claim 1 wherein the indenter is the shape of either a rod or a pointed cone.

10. A medical toilet comprising:
a. a toilet seat;
b. at least one fluid-filled bubble disposed on at least one surface of the medical toilet, wherein each of the at least one fluid-filled bubble protrudes from a surface of the medical toilet; and
c. a pressure sensor disposed on each of the at least one fluid-filled bubble;
d. wherein the pressure sensor is configured to measure a hardness of a soft tissue.

11. The medical toilet of claim 10, wherein the fluid comprises a gas.

12. The medical toilet of claim 10, wherein the fluid comprises a liquid.

13. The medical toilet of claim 10, wherein the at least one fluid-filled bubble comprises a plurality of fluid-filled bubbles.

14. The medical toilet of claim 13, wherein the plurality of fluid-filled bubbles is arranged in an array.

15. The medical toilet of claim 13, wherein the surface of the medical toilet comprises a surface of the toilet seat.

16. The medical toilet of claim 10, wherein the surface of the medical toilet comprises a surface of the toilet seat.

17. The medical toilet of claim 10, further comprising a toilet lid, wherein the surface of the medical toilet comprises a surface of the toilet lid.

18. The medical toilet of claim 10, further comprising a foot scale, wherein the surface of the medical toilet comprises a surface of the foot scale.

19. The medical toilet of claim 10, further comprising a controller, wherein the controller is in electronic connection with the pressure sensor on each of the at least one fluid-filled bubbles; and wherein the controller comprises non-transitory computer readable media, the non-transitory computer readable media comprising instructions for storing and analyzing measurements collected by the pressure sensor to determine the hardness of a soft tissue.

20. A medical toilet comprising:
a. a toilet seat;
b. an inflatable tube, the inflatable tube disposed on or in the toilet seat;
c. a pump, wherein the pump is in fluid communication with the inflatable tube;
d. a plurality of pressure sensors, the plurality of pressure sensors disposed along the inflatable tube;
e. a controller, wherein the controller is in electronic communication with the pump and the plurality of pressure sensors.
